(19) United States
(12) Patent Application Publication
(10) Pub. No.: US 2007/0014862 A1
(43) Pub. Date: Jan. 18, 2007

(54) Device for treating wound gaps

(76) Inventors: Cornelis Pameijer, Simsbury, CT (US); Steven Jensen, South Jordan, UT (US); Shaneen Wintch, Salt Lake City, UT (US)

Correspondence Address: MICHAUD-DUFFY GROUP LLP, 306 INDUSTRIAL PARK ROAD, SUITE 206, MIDDLETOWN, CT, 06457, US

(21) Appl. No.: 11/489,107

(22) Filed: Jul. 18, 2006

Related U.S. Application Data
(63) Continuation-in-part of application No. 10/961,604, filed on Oct. 12, 2004.

(51) Int. Cl. A61K 9/14

(52) U.S. Cl. 424/488

(57) ABSTRACT

A hemostatic agent comprises oxidized cellulose in the form of a compressible, shapeable mass that can remain substantially in the compressed or shaped form for placement on a bleed site or into a wound gap. The oxidized cellulose may be a pellet of unwoven oxidized cellulose fibrous strands, or it may be strands of unwoven cellulose fibers woven or otherwise arranged into a gauze or mesh. In a method of causing hemostasis, oxidized cellulose is provided in pellet form and applied to a wound gap. The pellet may be compressed before being applied to the wound, which thereby allows the pellet to expand to conform to the shape of the wound gap. The pellet may be allowed to remain in the wound gap during the healing of the wound, thus causing the pellet to be absorbed by the biological processes of the body.

### CROSS REFERENCE TO RELATED APPLICATION

This application is a continuation-in-part of U.S. patent application Ser. No. 10/961,604, filed Oct. 12, 2004, the contents of which are incorporated herein by reference in their entirety.

### BACKGROUND OF THE INVENTION

Blood is a liquid tissue that includes red cells, white cells, corpuscles, and platelets dispersed in a liquid phase. The liquid phase is plasma, which includes acids, lipids, solublized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

Medical, dental, and veterinary practitioners often encounter and/or treat patients with bleeding wounds, these wounds typically being caused by accidents or occurring as the result of surgical procedures. The principal method of treating these wounds is to stop the flow of blood, which generally involves applying pressure with a bandage to facilitate the formation of a clot. This is usually followed by protecting the clot from being prematurely dislodged and preventing the ingress of foreign bodies that would cause disease. Surface bandages or dressings are usually used for such purposes.

The treatment of the wound may also involve providing some type of aid that encourages the tissue to close over the wound, e.g., pulling the tissues adjacent the wound together and suturing, stapling, or otherwise causing them to remain closed over the wound. There are some wounds, however, in which suturing or stapling is not feasible or not practical. The result is a wound having a gap or void in soft tissue. This type of wound is not generally amenable to being sutured because there are not two soft tissue surfaces that can be pulled together and united.

The body's method of repairing open wounds is to fill them with blood that eventually coagulates to form a soft plug or coagulum. If this soft plug is left undisturbed, the wound will eventually heal. The material of the soft plug forms a barrier of cells that inhibit the ingress of bacteria, thus preventing infection, and is also vital in the process of cell replacement during the formation of new soft tissue. The body's tendency is to repair the wound socket with new soft tissue, but if the soft plug were to be dislodged before the wound fully healed a problem known as “dry socket” occurs. A dry socket is a gap from which the soft plug has been removed. The resulting hole eventually heals over.

In addition to dislodging the coagulum plug to create dry sockets, open wound gaps create a variety of other potential problems particularly in the oral environment. Where a soft plug has been formed, dental practitioners often encounter difficulty because the soft plug is so easily dislodged and removed by ordinary events such as chewing, drinking, sucking on a straw, salivating, etc. A bleeding gap left unfilled is also an ideal place for the compaction of food while eating. Bacteria thrive in any oral cavity, and an open wound filled with food becomes a breeding ground for infection. The primary manner of dealing with this problem has heretofore been to keep this area clean without disturbing the newly formed coagulated soft plug. In order to encourage their patients to keep these areas clean, dental practitioners often provide squirt bottles as a practical means of removing any debris. Patient compliance is a big factor in the success of such regimes, and failure to adequately remove debris, failure to execute constant vigilance in order to avoid dislodging the newly formed soft plug, or can result in infection. Even patients who diligently maintain cleaning regimes are still at risk for infections.

In order to absorb blood and facilitate the formation of a coagulum in an oral wound, the medical company Upjohn markets a “sterile absorbable gelatin sponge” called GEL FOAM, which comes in flat sheets. The product has the added advantage of being physiologically absorbed by the body in the event the material becomes trapped inside healing tissues. The disadvantage of GEL FOAM is that it exhibits a lack of physical cohesion, which therefore makes it unable to sufficiently withstand the oral environment. The GEL FOAM product is made from gelatin, a digestible foodstuff. Once placed into the oral environment, it is broken down by saliva like any other food. When contacted by the fluids of bleeding tissues, the GEL FOAM converts to a slimy gel, which acts almost like a lubricant on the surface of bleeding tissues. The resulting gel foam plug is also so delicate that it is easily displaced by physical means such as eating or brushing the teeth. In the oral environment, a GEL FOAM coagulated plug is not an improvement over the healing process of the body.

Practitioners in any medical field also routinely apply gauze to stop the flow of blood. Gauze, however, is designed to treat surface wounds and not to fill in voids. A sheet of gauze is impractical when attempting to fill a wound gap because the gauze must be methodically tucked into the gap or rolled into a ball prior to being tucked into the gap. Additionally, gauze in sheet form retains its elastic qualities and resists attempts to be forced into the shape necessary to fill a wound cavity. A sheet of gauze forced into a ball would begin to open when the distorting force was removed. This distorting force would be cumbersome to medical practitioners because (coupled with difficult-to-access wound sites) it generally poses a difficulty in getting the gauze properly placed in the wound. Furthermore, a flat sheet is not ideal for packing a socket because of a loss of compressibility and control during placement. Moreover, folding a sheet into a ball or the like prior to placement or randomly stuffing the sheet directly into a socket introduces air pockets that detract from the most useful positioning of the material. In addition, gauze is not absorbed into the body and therefore must eventually be removed.

What is needed is a device that can be placed to fill wound gaps with sufficient material cohesion and hemostatic (blood clotting) properties in order to create a more solid and retentive coagulum plug. This device must also remain during the healing cycle and be ultimately absorbed by physiological processes back into the tissues.

### SUMMARY OF THE INVENTION

Disclosed herein are hemostatic agents and devices for the treatment of wounds such as surface wounds and wound gaps. In surface wounds, a material capable of creating local hemostasis is brought into contact with bleeding tissue. In the case of wound gaps, the wound gap is packed with the material that is capable of creating the local hemostasis to aid in the formation of a solid and retentive coagulum plug. The material also can remain in the coagulum plug throughout the healing process and eventually be absorbed by physiological processes back into the tissues. It warrants that devices made of this material and used to fill a wound gap be in a form or shape as to aid the practitioner in filling or packing the wound gap.

In a first aspect, the present invention is directed to a hemostatic agent comprising oxidized cellulose in the form of a compressible, shapeable mass that, once compressed or shaped, remains substantially in the compressed or shaped form for placement on a bleed site or into a wound gap. The oxidized cellulose may be a pellet of unwoven oxidized cellulose fibrous strands, or it may be strands of unwoven cellulose fibers woven or otherwise arranged into a gauze or mesh. When inserted into a wound gap, the oxidized cellulose is able to expand to fill the wound gap upon releasing the compression forces.

In a second aspect, the present invention is directed to devices for promoting hemostasis, namely, oxidized cellulose in various forms, namely, compressible pellets and woven meshes or gauzes. The oxidized cellulose comprises non-woven strands of cellulose fiber, which may be cotton. The oxidized cellulose is manufactured by the action of nitrogen dioxide gas on the fiber. The nitrogen dioxide gas may be generated by (1) the catalytic reaction of manganese dioxide on nitric acid; (2) the catalytic reaction of manganese disulfide on nitric acid; or (3) the reaction of formaldehyde on nitric acid.

In a third aspect, the present invention is directed to a method of causing hemostasis. In the method, oxidized cellulose is provided in pellet form and applied to a wound gap. The pellet may be compressed before being applied to the wound, which thereby allows the pellet to expand to conform to the shape of the wound gap. The pellet may be allowed to remain in the wound gap during the healing of the wound, thus causing the pellet to be absorbed by the biological processes of the body.

Oxidized cellulose pellets applied to a wound or packed into a wound gap will immediately control local bleeding and form a solid coagulum plug with retention that is superior to current treatments. One advantage of the present invention is that the superior compressibility of the oxidized cellulose pellets allows the coagulum plug to be retained in the wound with the maximum contact with the adjacent tissue. The compressibility of the pellet facilitates the exertion of an outward force that retains the plug and the tissue in the proper positions to ensure a successful treatment.

Another advantage is that the pellet can remain in the body during the entire healing process. Because the oxidized cellulose is biocompatible with living tissue, the material in any form along with the resulting coagulum plug will eventually be absorbed by physiological processes of the body.

### DESCRIPTION OF THE DRAWINGS

FIG. 1 is a photograph of unwoven cellulose strands taken by a scanning electron microscope.

### DETAILED DESCRIPTION OF THE PREFERRED EMBODIMENTS

Disclosed herein are compositions and devices directed to the clotting of blood and the dressing of wounds. The compositions generally comprise oxidized cellulose materials that can minimize or stop the flow of blood by absorbing at least portions of the liquid phases of the blood, thereby promoting clotting. Although the compositions and devices are particularly suited for use in treating wound gaps in oral environments, the present invention is not limited in this regard and the compositions and devices can be used in any medical application in which it is desired to arrest the flow of blood.

Oxidized cellulose is a chemically oxidized form of a common cellulose fiber such as cotton and is also known as cellulosic acid, absorbable cellulose, or polyanhydroglucuronic acid. The degree of oxidation of the fiber is a function of the carboxylation content of the fibrous cellulose material. In particular, as the number of carboxyl groups on the cellulose structure is increased, the oxidation content correspondingly increases.

Oxidized cellulose may be manufactured by the action of nitrogen dioxide gas (NO₂) on cellulose fiber. Other methods of manufacturing oxidized cellulose include oxidation of cellulose fiber with aqueous oxidizing agents such as hypochlorite salts, although the use of such agents is less preferred than the use of nitrogen dioxide gas.

One method of generating nitrogen dioxide gas is by the catalytic reaction of manganese dioxide or manganese disulfide on concentrated nitric acid. Any amount of nitrogen dioxide can be generated by the metered addition of nitric acid to the manganese dioxide or manganese disulfide catalyst. In such a reaction, dinitrogen tetroxide (N₂O₄), which is a dimer of nitrogen oxide, is also formed in addition to the nitrogen dioxide. The formation of the dimer does not have an interfering effect on the oxidation of the cellulose.

In this method of nitrogen dioxide generation, unaltered cellulose fibers are introduced into a reaction vessel, and concentrated nitric acid is metered into a second enclosed vessel containing manganese dioxide powder. Nitrogen dioxide gas is evolved, which is piped to the reaction vessel containing the cellulose fibers. Once the nitrogen dioxide gas is piped to the reaction vessel containing the cellulose fibers, the reaction vessel is purged with an excess amount of nitrogen dioxide and left sealed for 30-45 days. This may alternatively be done in a pressurized environment of nitrogen dioxide. The oxidized cellulose is then removed and washed in dilute sodium bicarbonate solution, followed by multiple agitated rinses with distilled water. Alternatively, the oxidized cellulose may be degassed using other suitable means. The resulting oxidized cellulose is thus sufficiently carboxylated to provide a desirable hemostatic effect on a bleeding wound. The resulting fibers can also be autoclaved before use.

Another method of generating nitrogen dioxide gas is by the reaction of formaldehyde with concentrated nitric acid. This reaction, however, is not catalytic. In particular, formaldehyde is consumed in the reaction and is thus depleted. The formaldehyde readily reacts with the nitric acid to generate the nitrogen dioxide and the dimer. Again, the nitrogen dioxide gas is piped to the reaction vessel containing the cellulose fibers, and the reaction vessel is purged with excess nitrogen dioxide and sealed. The oxidized cellulose is removed, washed, and rinsed.

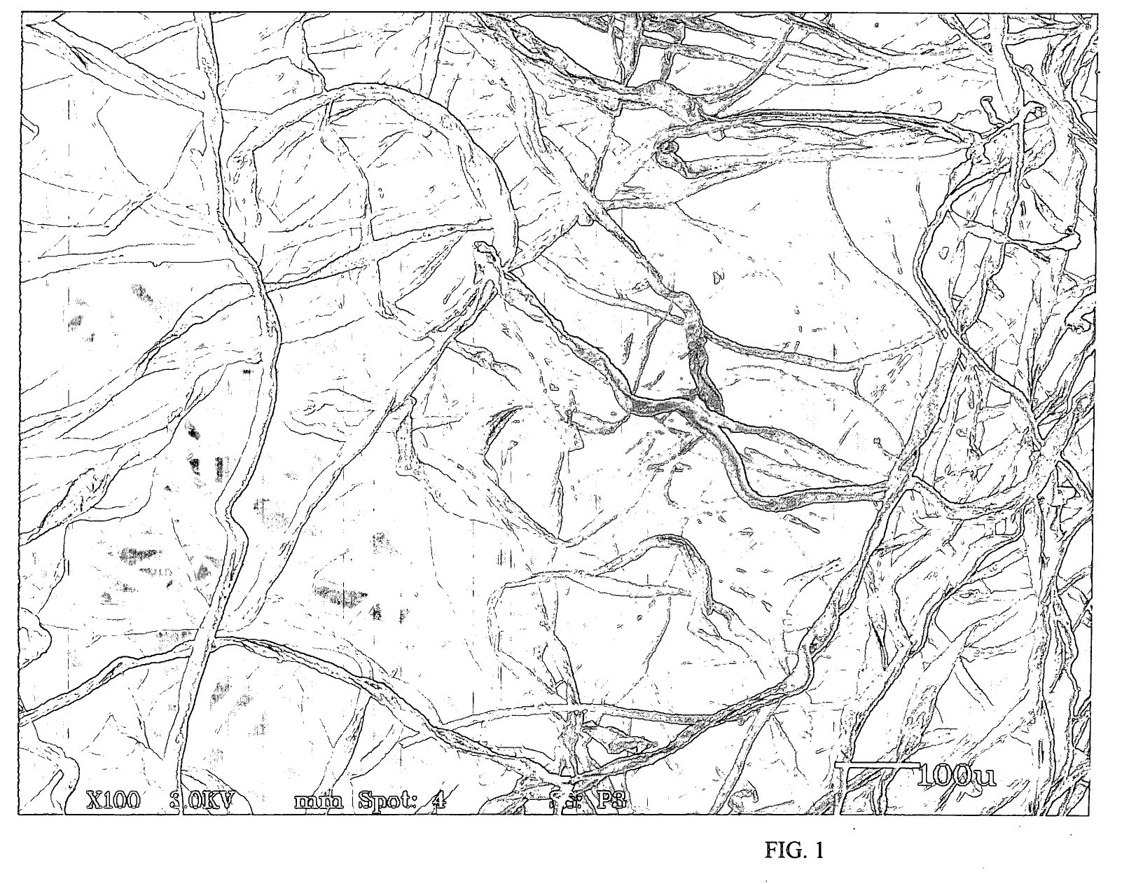

Referring to FIG. 1, the oxidized cellulose generated by either method is a mass of unwoven cellulose strands. The strands are loosely intermingled and easily compressed. The interstices between adjacent strands define areas in which the blood collects and the solids thereof agglomerate to facilitate the formation of clots. The compressibility of the unwoven cellulose strand mass allows the material to be formed into pellets. Other forms of the oxidized cellulose, such as those in woven form, are within the scope of the invention and suitable for use as gauze or mesh pads.

The quality of oxidation of the cellulose material can be determined by including a cotton string of known strength during the manufacture of the oxidized cellulose. The strength of the string is determined before it is included in the manufacturing process. One method of determining the strength of the string involves attaching a piece of the string to span between two points (e.g., a span of about 3 inches to about 4 inches), incrementally adding weight to the center point of the span, and noting the amount of weight required to cause the string to break. A mean value is obtained over about 4 or 5 trials and utilized to establish a baseline. In another method, the strength of the string can be determined via a pull test using an Instron strength testing apparatus.

After determining the strength of the string, a length of this string is incorporated into the material being treated to become oxidized cellulose. After completion of the treatment process and further upon completion of analysis of the desired properties of the oxidized cellulose, the strength test of the string is repeated. A mean value is obtained over about 4 or 5 trials and compared to the strength of the string before being incorporated into the material being treated to become oxidized cellulose. Subsequent production batches can be made to include the same (untreated) string material, which should be tested after completion of the treatment process. Upon testing the oxidized cellulose, the weight to break the string incorporated into the oxidized cellulose is preferably within about 10% of the mean value of the untreated string.

The clinical indications of oxidized cellulose are maximized when the cellulose is compressed into pellet form. Oxidized cellulose is observed to be most useful for filling wound gaps when it is formed into a compressible pellet, which thereby allows it to be packed into a socket. Packing the material directly into the wound in such a manner helps to increase retention by exerting an outward pressure against the surrounding tissue. This is ideal when attempting to fill a wound gap and it is desired that the pellet remain in the socket throughout the healing regime.

Irrespective of the form, there are multiple clinical applications for the oxidized cellulose. It is especially indicated for dental applications, namely for treating tooth extraction sockets, periodontal surgery, apicoectomy cases, and in implant dentistry. In both medical and dental applications, it can also be utilized in to fill voids that result from cyst removal. In the medical field, pellets or mesh pads can be used for traumatic accidents to cause an immediate cessation of bleeding. In surgical applications, the devices can be used to control bleeding. The devices can also be used as or in conjunction with first aid applications, e.g., to address minor scratches, scrapes, lacerations, or other lesions of the skin to stem the flow of blood. It is also especially indicated for patients who have a tendency to profusely bleed such as hemophiliac patients or patients taking blood thinning medications. In veterinary practice, the devices can be especially useful in less-than-septic conditions, e.g., in barnyards, kennels, and the like. A myriad of other uses of this device will become apparent during routine use by medical, dental, and veterinary practitioners.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A hemostatic agent, comprising: oxidized cellulose in the form of a compressible, shapeable, mass that remains substantially in said compressed or shaped form for placement on a bleed site or into a wound gap.

2. The hemostatic agent of claim 1, wherein said oxidized cellulose is in strand form and unwoven.

3. The hemostatic agent of claim 1, wherein said oxidized cellulose is carboxylated fibrous cellulose material.

4. The hemostatic agent of claim 3, wherein said fibrous cellulose material is cotton.

5. The hemostatic agent of claim 1, wherein said oxidized cellulose is formed by exposing cellulose material to nitrogen dioxide gas.

6. The hemostatic agent of claim 1, wherein said oxidized cellulose is in pellet form for insertion into said wound gap.

7. The hemostatic agent of claim 6, wherein said oxidized cellulose is cotton.

8. The hemostatic agent of claim 1, wherein said oxidized cellulose is manufactured by the action of nitrogen dioxide gas with cellulose fiber.

9. The hemostatic agent of claim 8, wherein said nitrogen dioxide gas is generated by the catalytic reaction of manganese dioxide with nitric acid.

10. The hemostatic agent of claim 8, wherein said nitrogen dioxide gas is generated by the catalytic reaction of manganese disulfide with nitric acid.

11. The hemostatic agent of claim 8, wherein said nitrogen dioxide gas is generated by the reaction of formaldehyde with nitric acid.

12. A device for promoting hemostasis, said device comprising: oxidized cellulose in strand form woven into a mesh.

13. The device of claim 12, wherein said oxidized cellulose in strand form comprises non-woven strands of cellulose fiber.

14. A method of causing hemostasis in a wound gap, said method comprising the steps of:
providing oxidized cellulose in pellet form; and
applying said oxidized cellulose to said wound gap.

15. The method of claim 14, further comprising forming said pellet by compressing a sheet of oxidized cellulose to a desired pellet size.

16. The method of claim 15, further comprising allowing said compressed pellet to expand to conform to a shape defined by said wound gap.

17. The method of claim 14, further comprising leaving said oxidized cellulose pellet in said wound gap to be enclosed by tissue during healing, said oxidized cellulose being absorbed by said tissue.

18. A method of fabricating oxidized cellulose, said method comprising the steps of:
generating. nitrogen dioxide gas in a first vessel;
piping said nitrogen dioxide gas to a second vessel containing cellulose fibers;
purging said second vessel with an excess amount of said nitrogen dioxide gas;
sealing said second vessel and allowing said second vessel to remain sealed for a predetermined period of time to oxidize said cellulose fibers;
washing said cellulose fibers in dilute sodium bicarbonate solution; and
rinsing said cellulose fibers.

19. The method of claim 18, wherein said step of generating said nitrogen dioxide gas comprises the step of adding nitric acid to a catalyst selected from the group consisting of manganese dioxide and manganese disulfide.

20. The method of claim 18, further comprising the step of generating dinitrogen tetroxide.

21. The method of claim 18, wherein said step of washing said cellulose fibers includes removing said cellulose fibers from said second reaction vessel.

22. The method of claim 18, wherein said step of generating said nitrogen dioxide gas comprises the step of reacting formaldehyde with nitric acid.

23. A method of determining a quality of a process of oxidizing cellulose material, said method comprising the steps of:
providing a thread of a certain length;
determining a particular strength for said thread;
incorporating said thread into cellulose material;
oxidizing said cellulose material; and
determining a strength of said cellulose material.

24. The method of claim 23, further comprising establishing a baseline from a plurality of determinations of particular strengths for said thread.

25. The method of claim 24, further comprising comparing said strength of said cellulose material to said established baseline.
